(19) United States
(12) Patent Application Publication
(10) Pub. No.: US 2016/0090608 A1
(43) Pub. Date: Mar. 31, 2016

(54) METHODS AND APPARATUSES FOR TREATING BIOMASS

(71) Applicant: UOP LLC, DES PLAINES, IL (US)

(72) Inventors: Amar Anumakonda, Naperville, IL (US); Francis Stephen Lupton, Evanston, IL (US)

(21) Appl. No.: 14/496,125

(22) Filed: Sep. 25, 2014

(51) Int. Cl. C12P 5/02

(52) U.S. Cl. CPC C12P 5/023

(57) ABSTRACT

Methods and apparatuses for treating biomass are provided herein. In an embodiment, an exemplary method of treating biomass includes providing a biomass stream that includes a lipid component or a derivative thereof and a contaminant component that includes nitrogen, phosphorous, ammonia, or a combination thereof. The contaminant component is extracted from the biomass stream with a wash composition that includes water to produce a washed biomass stream that includes the lipid component and a waste water stream that includes the contaminant component. The waste water stream is contacted with a substrate that includes a bound microorganism to remove the contaminant component from the waste water stream.

### TECHNICAL FIELD

The technical field generally relates to methods and apparatuses for treating biomass, and more particularly relates to methods and apparatuses for remediating a waste water stream that contains contaminants from washing of the biomass.

### BACKGROUND

Growth of world energy demand has prompted widespread research and development to identify alternative energy sources for satisfying such demand. One such promising alternative energy source is biofuel, which is derived from renewable resources such as plant or animal material. Biofuels can be used as combustible fuels themselves, can be used as an additive component of a combustible fuels, or can be co-processed with other hydrocarbon sources, such as a petroleum-based source of hydrocarbons, to produce combustible fuels.

Various contaminant species that are present in the biomass have a negative impact on biofuel production. These contaminant species may include nitrogen and phosphorus-containing species that are problematic for conversion of the biomass to a biofuel. In particular, the nitrogen and phosphorus-containing species contained in various plant oils, such as algal oils, are poisonous to many catalysts that are used to convert the plant oils to biofuels. As such, the biomass is typically washed or treated with wash compositions that contain water to remove contaminant species therefrom that could impact downstream conversion of the biomass to biofuel. However, washing of the biomass produces significant amounts of waste water. The waste water is often expunged to a holding pond or subject to costly disposal. While membrane filtration techniques have been employed to remediate the waste water and separate the contaminants therefrom, membrane filters are prone to clogging due to high particulate content in the waste water resulting from washing of the biomass. Furthermore, potentially valuable components of the biomass may be lost during remediation of the waste water to separate the contaminant components therefrom.

Accordingly, it is desirable to provide methods and apparatuses for treating biomass to remove at least some of a contaminant component therefrom with a wash composition that includes water, and to separate the contaminant component from the waste water stream with potential for recovery of the contaminant component or products produced therefrom. Furthermore, other desirable features and characteristics of the present invention will become apparent from the subsequent detailed description of the invention and the appended claims, taken in conjunction with the accompanying drawings and this

### BACKGROUND OF THE INVENTION

### Brief Summary

Methods and apparatuses for treating biomass are provided herein. In an embodiment, an exemplary method of treating biomass includes providing a biomass stream that includes a lipid component or a derivative thereof and a contaminant component that includes nitrogen, phosphorous, ammonia, or a combination thereof. The contaminant component is extracted from the biomass stream with a wash composition that includes water to produce a washed biomass stream that includes the lipid component and a waste water stream that includes the contaminant component. The waste water stream is contacted with a substrate that includes a bound microorganism to remove the contaminant component from the waste water stream.

In another exemplary embodiment, a method of treating biomass includes providing a biomass stream that includes a contaminant component including nitrogen, phosphorous, ammonia, or a combination thereof. The biomass stream is combined with a wash composition that includes water to produce a biomass and wash water mixture. The biomass and wash water mixture is separated using a centrifuge to produce a washed biomass stream and a waste water stream that includes the contaminant component. The contaminant component from the waste water stream is converted into a biogas stream.

In another exemplary embodiment, an apparatus for treating biomass is provided. The apparatus includes a wash stage, a separation stage, and a bioreactor. The wash stage has the capacity to receive a biomass stream and a wash composition that includes water. The separation stage is in fluid communication with the wash stage for receiving a biomass and wash water mixture. The separation stage includes a centrifuge to separate the biomass and wash water mixture into a waste water stream and washed biomass stream. The bioreactor is in fluid communication with the separation stage for receiving the waste water stream. The bioreactor includes a bound microorganism for removing a contaminant component from the waste water stream.

### BRIEF DESCRIPTION OF THE DRAWINGS

The various embodiments will hereinafter be described in conjunction with the following drawing figures, wherein like numerals denote like elements, and wherein:

FIG. 1 is a block diagram of an apparatus and method for treating biomass in accordance with an exemplary embodiment; and

FIG. 2 is a schematic representation of a bioreactor that may be included in the apparatus of FIG. 1 in accordance with an exemplary embodiment.

### DETAILED DESCRIPTION

The following detailed description is merely exemplary in nature and is not intended to limit the invention or the application and uses of the invention. Furthermore, there is no intention to be bound by any theory presented in the preceding background or the following detailed description.

Embodiments of the present disclosure are generally directed to methods and apparatuses for treating biomass. In accordance with the methods and apparatuses, the biomass is treated to remove at least some of a contaminant component therefrom with a wash composition by combining the biomass and the wash composition to produce a biomass and wash water mixture and then separating the biomass and wash water mixture to produce a washed biomass stream and a waste water stream that includes the contaminant component. The wash composition, as referred to herein, includes water and optionally additional components that may be employed to remove the contaminant component from the biomass. The contaminant component, as referred to herein, includes any compound(s) present within the biomass that is/are desirable to remove from the biomass prior to converting the biomass to biofuel, whether due to fuel quality concerns, catalyst poisoning, or any other detrimental impact of the component on the conversion of biomass to biofuel. The contaminant component includes nitrogen, phosphorus, ammonia, or a combination thereof, and may further encompass any other component of the biomass, whether naturally occurring in the biomass or introduced into the biomass, that has a detrimental impact on fuel quality or on the conversion of the biomass to biofuel and that can be separated from the desired portions of the biomass through physical and/or chemical separation. The contaminant component is separated from the waste water stream through contact with a substrate that includes a bound microorganism, such as anaerobic and/or aerobic microorganisms that are capable of separating the nitrogen and phosphorus, as well as other chemical oxygen demand (COD) and biological oxygen demand (BOD) species from the waste water stream. Further, in embodiments, the contaminant component is converted to a biogas stream that may be employed as an energy source within the process or as a product stream, thereby enabling recovery of the contaminant component.

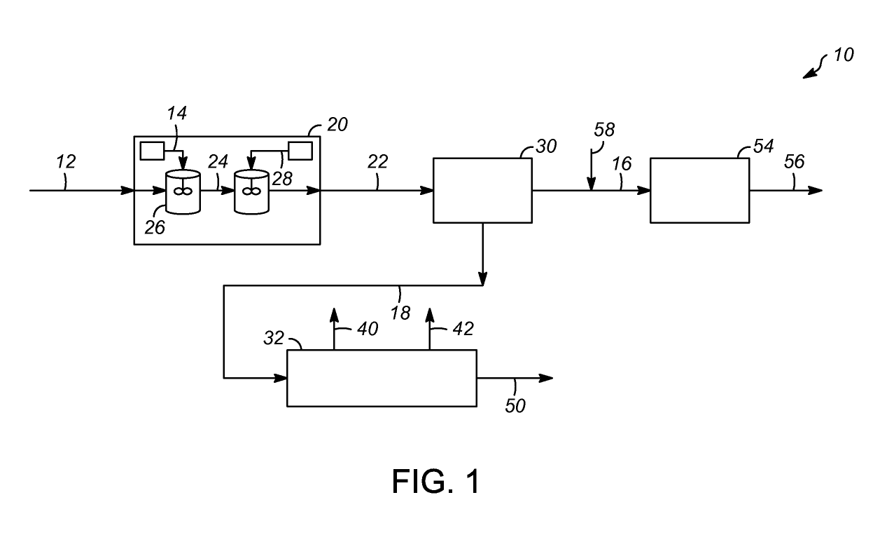

Referring to FIG. 1, a schematic depiction of an apparatus 10 for treating biomass in accordance with an exemplary embodiment is provided. A biomass stream 12 is provided that contains the contaminant component. In embodiments, the biomass stream 12 includes a lipid component or a derivative thereof. The lipid component may be separated from biomass and included in the biomass stream 12, may be derivatized from the biomass, or may be included in biomass that is directly included in the biomass stream 12. The lipid component or derivative thereof may be obtained from any natural oil or fat including, but not limited to, vegetable oils, tallows, animal fats, and the like. For example, in embodiments, the lipid component or derivative thereof is chosen from camelina oil, algal oil, pennycress oil, karanjia oil, palm stearin, fatty acid methyl ester, jatropha oil, cooking oil, palm fatty acid distillate, or a combination thereof. In specific embodiments, the biomass stream 12 includes algal oil, which may be crude or refined algal oil. Crude algal oil (e.g. “as extracted” algal oil) may be obtained via a mechanical, thermal, enzymatic, or chemical disruptive method that disrupts the algae cells to extract the algal oil through conventional techniques.

As set forth above, the contaminant component includes any component present within the biomass that is desirable to remove from the biomass prior to converting the biomass to biofuel, and the contaminant component includes nitrogen, phosphorus, ammonia, or a combination thereof. Additional contaminant species that may be included in the contaminant component may include chlorides or metals that are introduced into the biomass through fertilizers, pesticides, and the like. In embodiments, the contaminant component includes phospholipid, and may also include protein or nucleic acids in addition to or as an alternative to the phospholipid with the nitrogen and/or phosphorus present within the phospholipid, protein, and/or nucleic acids. Phospholipids are often present along with other lipids in biomass that contains the lipid component, and the phospholipids often may remain in the biomass stream 12 along with the lipid components described above.

In accordance with the exemplary method, the contaminant component is extracted from the biomass stream 12 with a wash composition 14 that includes water to produce a washed biomass stream 16 that includes the lipid component and a waste water stream 18 that includes the contaminant component. “Extracting”, as referred to herein, means that at least a portion of the contaminant component is separated from the biomass stream 12 through contact with a solvent (i.e., the wash composition 14) that is capable of taking up the at least a portion of the contaminant component in the biomass stream 12 and segregating the contaminant component from the biomass stream 12. In an embodiment and referring to FIG. 1, a wash stage 20 is provided that has the capacity to receive the biomass stream 12 and the wash composition 14. During washing, the biomass stream 12 is combined with the wash composition 14 in a wash unit 26 to produce a biomass and wash water mixture 22. In various embodiments, the wash unit 26 may be a batch unit or a continuous flow unit. Optionally, multiple wash units may be employed that combine the same or different wash composition with the biomass stream 12 to produce the biomass and wash water mixture 22, with the biomass and wash water mixture 22 as described herein including the biomass stream 12 and all wash compositions that are combined therewith during washing and prior to separation of the waste water stream 18 therefrom.

The wash composition 14 refers to any composition that contains water and that may be employed for washing the biomass stream 12 to remove at least some of the contaminant component therefrom. In embodiments, multiple different wash compositions are employed to wash the biomass stream 12. For example, in an embodiment and referring to FIG. 1, a multi-unit wash stage 20 may be employed. Conventional wash units 26 may be employed, and various different types of conventional washing unit operations may be conducted including, but not limited to, acid/base washing, digestion, contact washing, oil/water phase washing.

In an embodiment, acid/base washing is conducted through conventional techniques. For example, the biomass stream 12 and the wash composition 14 are combined to form a first intermediate stream 24. The wash composition 14 includes water and a base. In an exemplary embodiment, the wash composition 14 is a solution having a pH of about 10 or greater, such as about 10 to about 14. In one example, the wash composition 14 is a sodium hydroxide aqueous solution that has a pH of about 10 to about 12. The biomass stream 12 and the wash composition 14 are combined to form a first intermediate stream 24, which is then combined with a second wash composition 28 that includes acid to form the biomass and wash water mixture 22. In an exemplary embodiment, the second wash composition 28 includes water and an acid. In embodiments, sulfuric acid may be employed, with the acid is present in an amount of from about 1 to about 50 wt. %, for example from about 2 to about 50 wt. %, for example from about 5 to about 15 wt. %, such as about 10 wt. % of the second wash composition 28. In an exemplary embodiment, the second wash composition 28 has a pH of about 1 or less, for example from about 1 to about −1, for example from about 0 to about −0.5, such as about −0.1.

In accordance with the exemplary method, extraction proceeds by separating the biomass and wash water mixture 22 to produce the waste water stream 18 and the washed biomass stream 16. More specifically, a separation stage 30 is in fluid communication with the wash stage 20 for receiving the biomass and wash water mixture 22. Various techniques may be employed in the separation stage 30 to separate the biomass and wash water mixture 22 with the resulting waste water stream 18 including the contaminant component. Examples of suitable separation techniques include, but are not limited to, oil/water separation, sedimentation, filtration, centrifugation, flocculation, and froth floatation. In specific embodiments, the separation stage 30 includes a centrifuge to separate the biomass and wash water mixture 22 into the waste water stream 18 and the washed biomass stream 16. The lipid component or derivative thereof in the biomass and wash water mixture 22 may have a density similar to water, and centrifugation enhances separation of materials with similar densities due to increased centrifugal gravity whereas other settling methods may take too long to achieve effective separation at 1×gravity. Further, centrifugation is a low maintenance, low operating expense technique (generally involving a lesser number of process steps as compared to alternative separation techniques) and provides high throughput (due to continuous operation). In embodiments, the waste water stream 18 includes water in an amount of at least about 50 weight %, such as from about 80 to 99 weight %, such as from about 95 to about 99 weight %, with the balance of waste water stream 18 including the contaminant component and optionally other chemical species (e.g., salts of acids or bases) that may be produced during washing of the biomass stream 12.

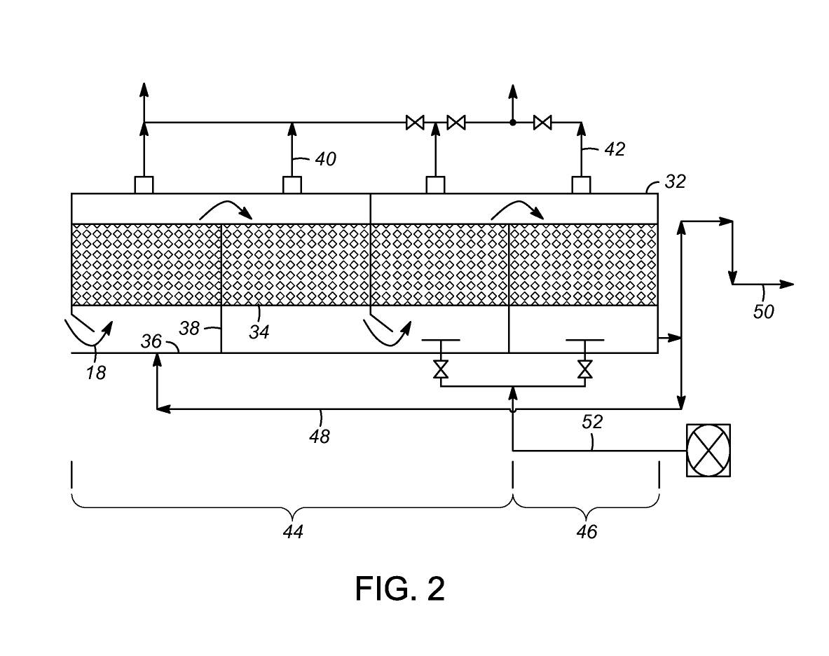

In accordance with the exemplary method, the contaminant component is separated from the waste water stream 18 to effectively remediate the waste water stream 18 and to produce a treated water stream 50. In embodiments, the contaminant component is separated by contacting the waste water stream 18 with a substrate that includes a bound microorganism to remove the contaminant component from the waste water stream 18. In an embodiment and referring to FIG. 1, a bioreactor 32 is in fluid communication with the separation stage 30 for receiving the waste water stream 18 for separating the contaminant component from the waste water stream 18. The bioreactor 32, as referred to herein, incorporates a microorganism to effect separation of the contaminant component from the waste water stream 18. For example and referring to FIG. 2, the bioreactor 32 is a fixed film bioreactor that includes the substrate 34 that includes the bound microorganism. In embodiments, the bioreactor 32 includes chambers 36 with baffles 38 separating the chambers 36 to promote plug flow of the waste water stream 18 through the bioreactor 32. In various embodiments, anaerobic and/or aerobic microorganisms may be employed within the bioreactor 32 to separate the species of the contaminant component from the waste water stream 18 and to consume and/or convert the species of the contaminant component into various products, with the bioreactor 32 operated at anaerobic and/or aerobic conditions at locations within the bioreactor 32 where the respective microorganisms are disposed. For example, an oxygen gas feed line 52 may be provided to feed oxygen into or upstream of regions of the bioreactor 32 where the aerobic microorganism is disposed. Depending upon the type of microorganism employed, different products are obtained from the microorganism. For example, the bound microorganism may be an anaerobic microorganism and contacting the waste water stream 18 with the substrate 34 that includes the anaerobic microorganism produces a biogas stream 40, which includes methane as well as carbon dioxide and other gaseous species. As another example, the bound microorganism may be an aerobic microorganism and contacting the waste water stream 18 with the substrate 34 that includes the aerobic microorganism produces exhaust gas 42 that includes nitrogen gas, among other gaseous species.

In embodiments and as shown in FIG. 2, both an anaerobic microorganism and an aerobic microorganism are employed, with the anaerobic microorganism bound on the substrate 34 in an anaerobic section 44 and the aerobic microorganism bound on the substrate 34 in an aerobic section 46. The waste water stream 18 is contacted with the substrate 34 that includes the anaerobic microorganism in the anaerobic section 44 to produce the biogas stream 40, and the waste water stream 18 is also contacted with the substrate 34 that includes the aerobic microorganism to produce exhaust gas 42 in the aerobic section 46. In embodiments and as shown in FIG. 2, the waste water stream 18 is sequentially contacted with the substrate 34 that includes the anaerobic microorganism in the anaerobic section 44 followed by contacting the waste water stream 18 with the substrate 34 that includes the aerobic microorganism in the aerobic section 46. However, it is to be appreciated that in other embodiments and although not shown, the order of the anaerobic section and the aerobic section may be reversed. In embodiments, ammonia is a species of the contaminant component that is present in the waste water stream 18. In this embodiment, ammonia is not consumed or converted by the anaerobic microorganism in the anaerobic section 44. However, the aerobic microorganism converts ammonia to nitrate, and contacting the aerobic microorganism with the waste water stream 18 that includes ammonia produces nitrate that can be reduced by the anaerobic microorganism to nitrogen gas. Thus, in an embodiment, nitrate 48 that is produced by the aerobic microorganism may be separated from the treated water stream 50 and recycled to contact the substrate 34 that includes the anaerobic microorganism in the anaerobic section 44 for conversion to nitrogen gas. In this regard, ammonia can effectively be removed from the waste water stream 18 and converted to inert nitrogen gas.

The biogas stream 40 that is produced by the anaerobic microorganism in the anaerobic section 44 of the bioreactor 32 may be employed for various downstream processes, including energy recovery. In embodiments, the wash composition 14 is heated using the biogas stream 40. However, it is to be appreciated that the biogas stream 40 may be employed in various applications. The exhaust gas 42 is generally vented to the ambient environment. The treated water stream 50 may be recycled for reuse in washing the biomass stream.

In embodiments, the treated water stream 50 contains significantly reduced amounts of ammonia, nitrogen, and phosphorous, as well as various BOD and COD species, as compared to the waste water stream 18. For example, in embodiments, BOD species are reduced by at least 80 weight %, such as at least 90 weight %, such as from about 95 to about 99 weight %, based on the total content of BOD species in the waste water stream 18. As another example, COD species are reduced by at least 70 weight %, such as at least 80 weight %, such as from about 80 to about 85 weight %, based on the total content of COD species in the waste water stream 18. As another example, ammonia content is reduced by at least 90 weight %, such as at least 95 weight %, such as from about 97 to about 99 weight %, based on the total content of ammonia in the waste water stream 18. As another example, total nitrogen content is reduced by at least 70 weight %, such as at least 75 weight %, such as from about 78 to about 85 weight %, based on the total content of all nitrogen-containing species in the waste water stream 18. As another example, total phosphorus content is reduced by at least 50 weight %, such as from about 50 to about 60 weight %, based on the total content of all phosphorus-containing species in the waste water stream 18.

In an embodiment, the washed biomass stream 16 is further processed for conversion to biofuel through conventional techniques. For example, in an embodiment and as shown in FIG. 1, the apparatus 10 includes a biomass conversion stage 54 that is in fluid communication with the separation stage 30 for receiving the washed biomass stream 16. In an embodiment, the washed biomass stream 16 that includes the lipid component is deoxygenated to form normal paraffins 56, and may optionally be hydrogenated prior to deoxygenation to saturate double bonds that may be present in the lipid component. Deoxygenation of biomass streams that include the lipid component is a conventional process. In particular, deoxygenating the washed biomass stream 16 includes reacting the washed biomass stream 16 with hydrogen 58 at elevated temperatures and pressures, for a specified period of time, in the presence of a deoxygenation catalyst to form normal paraffins 56. As referred to herein, deoxygenation refers to a unit operation by which oxygen is removed from compounds in the washed biomass stream 16 through any mechanism including, but not limited to, decarboxylation, decarbonylation, and/or hydrodeoxygenation. In an embodiment, hydrogen 58 is mixed with the washed biomass stream 16 prior to introduction into the biomass conversion stage 54. For certain applications, such as for renewable jet fuel, an additional cracking process may also be conducted in the biomass conversion stage 54 under severe conditions that promote both desired cracking/isomerization and undesired over-cracking reactions. The additional cracking process may be conducted in accordance with conventional techniques.

In alternative embodiments, biomass that includes lignocellulosic material is subject to treatment in accordance with methods described herein. The methods may be conducted in the apparatus 10 as described above and shown in FIG. 1, although different treatment techniques may be conducted in the various stages of the apparatus 10. For example, although biomass streams 12 that include lignocellulosic material are generally subject to washing with the wash composition 14 that includes water, such washing may be conducted at various locations that result in production of the waste water stream 18. For example, the waste water stream 18 may be produced during pre-wash or digestion of the biomass stream 12, with the same species described above for the contaminant component included in the waste water stream 18 among other possible contaminant species. The waste water stream 18 may be processed in the same manner as described above in the bioreactor 32. However, because lignocellulosic material is present in the washed biomass stream 16, alternative techniques to those described above are generally employed in the biomass conversion stage 54 for conversion of the washed biomass stream 16 to biofuel. For example, in an embodiment, the washed biomass stream 16 is pyrolyzed to produce a pyrolysis product stream 56. Pyrolysis is a commonly-used process for converting biomass into biofuel, and pyrolysis can be conducted through either a thermal process or a catalytic process. In the catalytic pyrolysis process, the washed biomass stream 16 is rapidly heated under an inert atmosphere in the presence of a catalyst, such as an acid or zeolitic catalyst, to promote deoxygenation and cracking of pyrolysis vapors into hydrocarbons and oxygen-containing compounds, such as phenol, cresol, and alcohols such as C1 to C4 alcohols.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention. It being understood that various changes may be made in the function and arrangement of elements described in an exemplary embodiment without departing from the scope of the invention as set forth in the appended claims.

What is claimed is:

1. A method of treating biomass, wherein the method comprises:
providing a biomass stream comprising a lipid component or a derivative thereof and a contaminant component including nitrogen, phosphorous, ammonia, or a combination thereof;
extracting the contaminant component from the biomass stream with a wash composition comprising water to produce a washed biomass stream comprising the lipid component and a waste water stream comprising the contaminant component; and
contacting the waste water stream with a substrate comprising a bound microorganism to remove the contaminant component from the waste water stream.

2. The method of claim 1, wherein the bound microorganism is an anaerobic microorganism and wherein contacting the waste water stream comprises contacting the waste water stream with the substrate comprising the anaerobic microorganism to produce a biogas stream.

3. The method of claim 2, further comprising heating the wash composition using the biogas stream.

4. The method of claim 1, wherein the bound microorganism is an aerobic microorganism and wherein contacting the waste water stream comprises contacting the waste water stream with the substrate comprising the aerobic microorganism to produce exhaust gas.

5. The method of claim 1, wherein the bound microorganism comprises an anaerobic microorganism and an aerobic microorganism, and wherein contacting the waste water stream comprises contacting the waste water stream with the substrate comprising the anaerobic microorganism to produce a biogas stream and contacting the waste water stream with the substrate comprising the aerobic microorganism to produce exhaust gas.

6. The method of claim 5, wherein contacting the waste water stream with the substrate comprising the anaerobic microorganism and contacting the waste water stream with the substrate comprising the aerobic microorganism comprises sequentially contacting the waste water stream with the substrate comprising the anaerobic microorganism followed by contacting the waste water stream with the substrate comprising the aerobic microorganism.

7. The method of claim 6, wherein contacting the waste water stream with the substrate comprising the aerobic microorganism comprises contacting the waste water stream comprising ammonia with the substrate comprising the aerobic microorganism to produce nitrate.

8. The method of claim 7, further comprising recycling the nitrate to contact the substrate comprising the anaerobic microorganism.

9. The method of claim 1, wherein providing the biomass stream comprises providing the biomass stream comprising the lipid component or derivative thereof chosen from camelina oil, algal oil, pennycress oil, karanjia oil, palm stearin, fatty acid methyl ester, jatropha oil, cooking oil, palm fatty acid distillate, or a combination thereof.

10. The method of claim 9, wherein providing the biomass stream comprises providing the biomass stream comprising algal oil.

11. The method of claim 1, wherein providing the biomass stream comprises providing the biomass stream comprising the contaminant component including phospholipid.

12. The method of claim 1, wherein extracting the contaminant component comprises separating the waste water stream from the washed biomass stream through centrifugation.

13. The method of claim 1, wherein extracting the contaminant component comprises combining the biomass stream and the wash composition comprising water and a base to form a first intermediate stream.

14. The method of claim 13, wherein extracting the contaminant component further comprises combining the first intermediate stream and a second wash composition comprising acid to form a biomass and wash water mixture.

15. The method of claim 1, further comprising deoxygenating the washed biomass stream to form normal paraffins.

16. A method of treating biomass, wherein the method comprises:
providing a biomass stream comprising a contaminant component including nitrogen, phosphorous, ammonia, or a combination thereof;
combining the biomass stream with a wash composition comprising water to produce a biomass and wash water mixture;
separating the biomass and wash water mixture using a centrifuge to produce a washed biomass stream and a waste water stream comprising the contaminant component;
converting the contaminant component from the waste water stream into a biogas stream.

17. The method of claim 16, wherein providing the biomass stream comprises providing the biomass stream comprising lignocellulosic material.

18. The method of claim 16, wherein the biomass stream and the wash composition are combined during digestion.

19. The method of claim 16, further comprising pyrolyzing washed biomass from the washed biomass stream to produce a pyrolysis product stream.

20. An apparatus for treating biomass, wherein the apparatus comprises:
a wash stage having the capacity to receive a biomass stream and a wash composition comprising water;
a separation stage in fluid communication with the wash stage for receiving a biomass and wash water mixture, wherein the separation stage comprising a centrifuge to separate the biomass and wash water mixture into a waste water stream and washed biomass stream;
a bioreactor in fluid communication with the separation stage for receiving the waste water stream, wherein the bioreactor comprises a bound microorganism for removing a contaminant component from the waste water stream.
